(19) United States
(12) Patent Application Publication
(10) Pub. No.: US 2010/0130740 A1
(43) Pub. Date: May 27, 2010

(54) PROCESS FOR PREPARATION OF RISPERIDONE

(76) Inventors: Guiling Zhang, Shandong (CN); Yidong Zhu, Shandong (CN); Chuanwen Fan, Shandong (CN); Minghui Zhang, Shandong (CN); Jingyi Wang, Shandong (CN)

Correspondence Address: DAVID AND RAYMOND PATENT FIRM, 108 N. YNEZ AVE., SUITE 128, MONTEREY PARK, CA, 91754, US

(21) Appl. No.: 12/452,874

(22) PCT Filed: Jul. 25, 2008
(86) PCT No.: PCT/CN2008/071754

§ 371 Date: Jan. 25, 2010

(30) Foreign Application Priority Data
Jul. 26, 2007 (CN) 200710016232.6

(51) Int. Cl. C07D 471/04

(52) U.S. Cl. 544/282

(57) ABSTRACT

The invention provides a more handy and environment friendly process for preparing risperidone, which comprises reacting 6-fluoro-3-(4-piperidinyl)-1,2-benzisoxazole hydrochloride with 3-(2-chloroethyl)-2-methyl-6,7,8,9-tetrahydro-4H-pyrido[1,2-a]pyrimidin-4-one in a basic aqueous solution or suspension. The concentration of alkaline metal carbonates in the basic aqueous solution or suspension is ranged from 15% to 40%. The reaction is carried out at a temperature range of 101-140° C. and the reaction is completed within 10 minutes to 2 hours.

### BACKGROUND OF THE PRESENT INVENTION

1. Field of Invention

The present invention is related to new processes for the preparation of risperidone3-[2-[4-(6-Fluoro-1,2-benzisoxazol-3-yl)piperidin-1-yl]ethyl]-2-methyl-6,7,8,9-tetrahydro-4H-pyrido[1,2-a]pyrimidin-4-one which is used for the treatment of schizophrenia and psychotic disorder. The present invention belongs to the pharmaceutical chemical field.

2. Description of Related Arts

Risperidone, the structure is shown in Formula (I), the chemical name is 3-[2-[4-(6-Fluoro-1,2-benzisoxazol-3-yl)piperidin-1-yl]ethyl]-2-methyl-6,7,8,9-tetrahydro-4H-pyrido[1,2-a]pyrimidin-4-one.

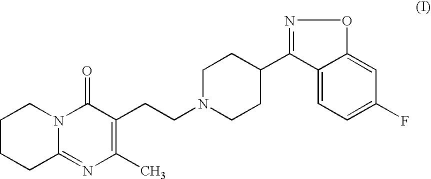

Risperidone is a selective monoaminergic antagonist belonging to benzisoxazole derivatives. Risperidone has a high affinity for both serotonergic 5-HT₂ and dopaminergic D₂ receptors. It also binds to α1-adrenergic receptors and with lower affinity to H₁-histaminergic and α2-adrenergic receptors. Risperidone has no affinity for cholinergic receptors. As a potent D2 antagonist, risperidone is good for the positive symptoms of schizophrenia, causing lower adverse effect of motor inhibition and tonic syncope than that of typical neuroleptics cause. It could reduce the extrapyramidal side effects through the action at 5HT and dopamine antagonist, and extend its therapeutic action to negative symptoms and emotional symptoms of schizophrenia. Risperidone of “Johnson & Johnson”, which is came into market in the USA in 1993, and launched in dozens of countries now. The global sales are about up to 4.76 billion dollars by March 2007.

Several chemical processes for the preparation of the risperidone have been developed, of which the synthesis using 3-(2-substituted ethyl)-2-methyl-6,7,8,9-tetrahydro-4H-pyrido[1,2-a]pyrimidin-4-one of formula (II) and 6-fluoro-3-(4-piperidinyl)-1,2-benzisoxazole of formula (III) is preferred for industrial application.

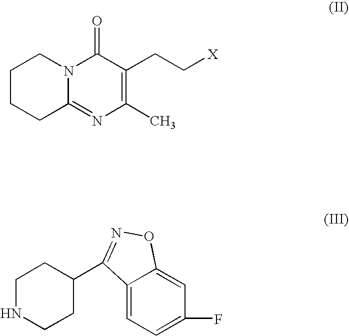

wherein X represents halogen.

U.S. Pat. No. 4,804,663, the inventor Ludo E. J. Kennis etc., describes a process for the risperidone, as shown in Route 1:

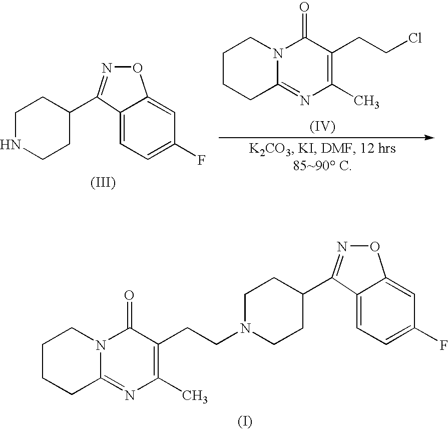

According to the U.S. Pat. No. 4,804,663 description the preparation of risperidone by condensing of 6-fluoro-3-(4-piperidinyl)-1,2-benzisoxazole of formula (III) with 3-(2-chloroethyl)-2-methyl-6,7,8,9-tetrahydro-4H-pyrido[1,2-a]pyrimidin-4-one of formula (IV) in the presence of potassium carbonate as a base and potassium iodide as a catalyst in dimethylformamide (DMF) medium at 85-90° C., and the reaction time takes 12 hours. The preparation of risperidone in this way must be carried out in the presence of a catalyst and a inorganic base in a organic solvent medium, the reaction time takes 12 hours, the overall yield as described here is 46%. Therefore, this is a complex process, the operation cycle is long, and the yield is low.

CN1984913, the inventor CZIBULSA, Laszlo etc., describes another process for producing risperidone, as shown in Route 2:

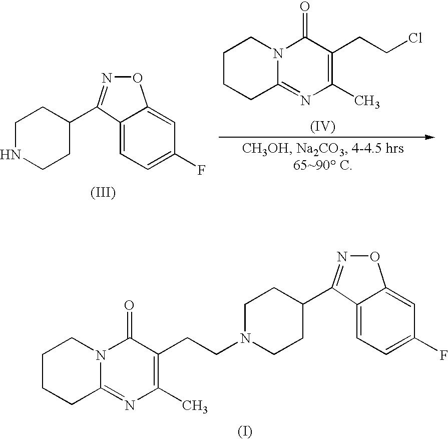

According to the CN1984913 description the risperidone is prepared by condensation of 6-fluoro-3-(4-piperidinyl)-1,2-benzisoxazole of formula (III) with 3-(2-chloroethyl)-2-methyl-6,7,8,9-tetrahydro-4H-pyrido[1,2-a]pyrimidin-4-one of formula (IV) in the presence of an acid binding agent in absolute methanol medium at 65-90° C., and the reaction time takes 4-4.5 hours. The preparation of risperidone in this way must be carried out in a methanol medium which is neurotoxicity, and it is not friendly for the worker and environment.

WO2005030772, the inventor Srinvasa Rao, Guntu etc., describes another process for producing risperidone, as shown in Route 3:

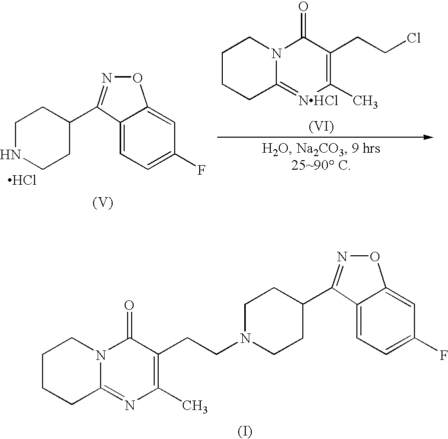

According to the WO2005030772 description the risperidone is prepared by condensing of 6-fluoro-3-(4-piperidinyl)-1,2-benzisoxazole hydrochloride of formula (V) with 3-(2-chloroethyl)-2-methyl-6,7,8,9-tetrahydro-4H-pyrido[1,2-a]pyrimidin-4-one hydrochloride of formula (VI) in a mixture of basic aqueous solution and water-soluble organic solvents or in basic aqueous solution. Adding the reactant and reagent must be in sequential for the synthetic process. 3-(2-chloroethyl)-2-methyl-6,7,8,9-tetrahydro-4H-pyrido[1,2-a]pyrimidin-4-one hydrochloride (VI) must be prepared into solution and be added slowly in a period of over 5 hours. After adding it the reaction must be carried out at a period of 4-5 hours at the temperature ranging from 25° C. to 90° C. The overall yield is about 60%. Therefore, this is a complex process, the operation cycle is long, and it is not appropriate to industrial production.

### SUMMARY OF THE PRESENT INVENTION

To overcome the drawbacks of existing preparing processes, the present invention provides a more handy and environment friendly process for preparing risperidone.

The preparing method provided by the present invention comprises reacting 6-fluoro-3-(4-piperidinyl)-1,2-benzisoxazole hydrochloride of formula (V) with 3-(2-chloroethyl)-2-methyl-6,7,8,9-tetrahydro-4H-pyrido[1,2-a]pyrimidin-4-one of formula (IV) which are commercially available in a environment friendly basic aqueous solution or suspension to obtain crude risperidone rapidly and efficiently, then the crude product is purified with any known and common method. The purity of the risperidone is more than 99%.

The present invention provides a method for preparing risperidone of formula (I), which comprises the process of refluxing the reaction 6-fluoro-3-(4-piperidinyl)-1,2-benzisoxazole hydrochloride of formula (V) with 3-(2-chloroethyl)-2-methyl-6,7,8,9-tetrahydro-4H-pyrido[1,2-a]pyrimidin-4-one of formula (IV) in a basic aqueous solution or suspension to obtain crude risperidone.

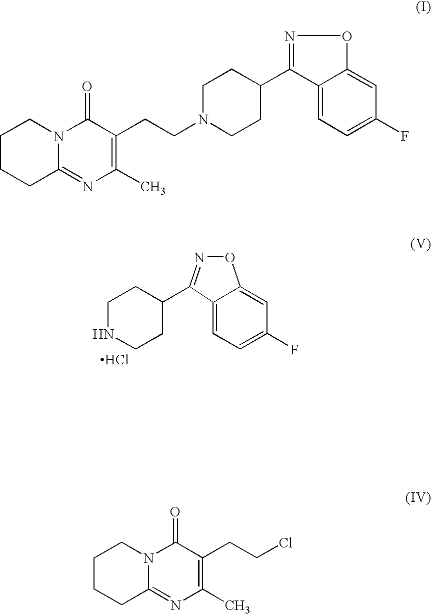

The method for preparing risperidone described in the present invention, the reacting molar ratio of 6-fluoro-3-(4-piperidinyl)-1,2-benzisoxazole hydrochloride of formula (V) and 3-(2-chloroethyl)-2-methyl-6,7,8,9-tetrahydro-4H-pyrido[1,2a]pyrimidin-4-one of formula (IV) is ranging from 1:1.0 to 1:2.0, preferably from 1:1.1 to 1:1.3.

According to the method for preparing risperidone of the invention, the basic aqueous solution or suspension is an aqueous solution or suspension of alkaline metal carbonates.

According to the method for preparing risperidone of the invention, the content of alkaline metal carbonates in the basic aqueous solution or suspension is ranging from 15% to 40% (weight %), preferably 25% (weight %).

According to the method for preparing risperidone of the invention, the alkaline metal carbonates in the basic aqueous solution or suspension is selected from sodium carbonate, potassium carbonate and a mixture thereof, preferably sodium carbonate.

According to the method for preparing risperidone of the invention, the basic aqueous solution or suspension is employed in an amount ranging from 5 ml to 18 ml, preferably from 7 ml to 11 ml, based on per gram of 6-fluoro-3-(4-piperidinyl)-1,2-hydrochloride of formula (V).

According to the method for preparing risperidone of the invention, the reaction temperature is ranging from 101° C. to 140° C., preferably from 101° C. to 130° C., the reaction time is ranging from 10 min to 2 h, preferably from 15 min to 1 h.

In the existing technology for preparing of risperidone, the reported reaction temperature is not higher than 100° C., and the reaction is slow so the reaction time is very long. After many times experiments, the present inventor found that it can greatly accelerate the reaction by increasing the reaction temperature to 100° C. or even higher. In the present invention, 101-140° C. of the reaction temperature is adopted, and it did not produce abnormal side effects or increase the amount of impurities of risperidone, instead, it results in shorter reaction time, higher yield and lower cost, so it is very suitable for large-scale industrial production.

According to the preparing process of the invention, the reaction yield is nearly or even more than 90%, the product of risperidone can simply obtain a purity of more than 99.0% by recrystallization.

The raw materials of preparing process described in the present invention are commercially available, and do not need to be dealt with acids and alkalines and into the corresponding alkalines or salts. Water is used as a medium and no organic solvent is needed, so there is not adverse affect to the operator and the environment.

These and other objectives, features, and advantages of the present invention will become apparent from the following detailed description, the accompanying drawings, and the appended claims.

### DETAILED DESCRIPTION OF THE PREFERRED EMBODIMENT

The following examples are only used to further implement of the present invention. They do not limit the present invention.

### Example 1

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.30 g of 3-(2-chloroethyl)-2-methyl-6,7,8,9-tetrahydro-4H-pyrido[1,2-a]pyrimidin-4-one are placed, then add a sodium carbonate solution or suspension (dissolved or suspended 4.5 g of sodium carbonate in 25 ml water). The mixture is put into heating bath at 120-130° C. with stirring for 60 min, then cooled with continuous stirring to the room temperature and the precipitate solid is filtered, washed with pure water, and dried to give 3.70 g of the product in 90.2% yield. The product is purified to obtain a purity of 99.5% (determined by HPLC) with DMF and isopropanol.

¹H-NMR (DMSO-d₆, 400 MHz) δ 1.884 (m, 2H), 1.965 (m, 2H), 2.098 (sxt, 4H), 2.273 (q, 2H), 2.315 (s, 3H), 2.549 (q, 2H), 2.771 (q, 2H), 2.870 (t, 2H), 3.081 (m, 1H), 3.183 (d, 2H), 3.935 (t, 2H), 7.052 (ddd, 1H), 7.238 (dd, 1H), 7.711 (q, 1H).

### Example 2

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

The same method as described in Example 1 is followed, but a potassium carbonate solution or suspension (dissolved or suspended 4.5 g of potassium carbonate in 25 ml water) is used and the heating is carried out at 130-140° C. to give 3.64 g of the product. The yield is 88.8%. The product is purified to obtain a purity of 99.5% (determined by HPLC) with DMF and isopropanol.

### Example 3

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.30 g of 3-(2-chloroethyl)-2-methyl-6,7,8,9-tetrahydro-4H-pyrido[1,2-a]pyrimidin-4-one are placed, then a sodium carbonate solution or suspension (dissolved or suspended 8.5 g of sodium carbonate in 25 ml water) is added. The mixture is put into heating bath at 110-120° C. with stirring for 40 min, then cooled with continuous stirring to the room temperature and the precipitate solid is filtered, washed with pure water, and dried to give 3.78 g of the product in 92.2% yield. The product is purified to obtain a purity of 99.5% (determined by HPLC) with DMF and isopropanol.

### Example 4

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

The same method as described in Example 3 is followed, but potassium carbonate replaces sodium carbonate, and to get reaction at 130-140° C. to give 3.76 g of the product. The yield is 91.7%. The product is purified to obtain a purity of 99.5% (determined by HPLC) with DMF and isopropanol.

### Example 5

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.30 g of 3-(2-chloroethyl)-2-methyl-6,7,8,9-tetrahydro-4H-pyrido[1,2-a]pyrimidin-4-one are placed, then sodium carbonate solution or suspension (dissolved or suspended 16.5 g of sodium carbonate in 25 ml water) is added. The mixture is put into heating bath at 101-110° C. with stirring for 15 min, then cooled with continuous stirring to the room temperature and the precipitate solid is filtered, washed with pure water, and dried to give 3.61 g of the product in 88.0% yield. The product is purified to obtain a purity of 99.5% (determined by HPLC) with DMF and isopropanol.

### Example 6

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

The same method as described in Example 5 is followed, but potassium carbonate replaces sodium carbonate, and to get reaction at 110-120° C. to give 3.65 g of the product. The yield is 89.0%. The product is purified to obtain a purity of 99.5% (determined by HPLC) with DMF and isopropanol.

### Example 7

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.30 g of 3-(2-chloroethyl)-2-methyl-6,7,8,9-tetrahydro-4H-pyrido[1,2-a]pyrimidin-4-one are placed, then a carbonate solution or suspension (dissolved or suspended 8.5 g of sodium carbonate and 8.0 g of potassium carbonate in 25 ml water) is added. The mixture is put into heating bath at 110-120° C. with stirring for 20 min, then cooled with continuous stirring to the room temperature and the precipitate solid is filtered, washed with pure water, and dried to give 3.58 g of the product in 87.3% yield. The product is purified to obtain a purity of 99.5% (determined by HPLC) with DMF and isopropanol.

### Example 8

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.49 g of 3-(2-chloroethyl)-2-methyl-6,7,8,9-tetrahydro-4H-pyrido[1,2-a]pyrimidin-4-one are placed, then a sodium carbonate solution or suspension (dissolved or suspended 8.5 g of sodium carbonate in 25 ml water) is added. The mixture is put into heating bath at 110-120° C. with stirring for 40 min, then cooled with continuous stirring to the room temperature and the precipitate solid is filtered, washed with pure water, and dried to give 3.81 g of the product in 92.9% yield. The product is purified to obtain a purity of 99.5% (determined by HPLC) with DMF and isopropanol.

### Example 9

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.95 g of 3-(2-chloroethyl)-2-methyl-6,7,8,9-tetrahydro-4H-pyrido[1,2-a]pyrimidin-4-one are placed, then a sodium carbonate solution or suspension (dissolved or suspended 8.5 g of sodium carbonate in 25 ml water) is added. The mixture is put into heating bath at 110-120° C. with stirring for 40 min, then cooled with continuous stirring to the room temperature and the precipitate solid is filtered, washed with pure water, and dried to give 3.82 g of the product in 93.2% yield. The product is purified to obtain a purity of 99.5% (determined by HPLC) with DMF and isopropanol.

### Example 10

### Preparation of 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one (risperidone)

In a 50-ml reaction flask, 2.56 g of 6-fluoro-3-(4-piperidinyl)-1,2-benzisoxazole hydrochloride and 2.30 g of 3-(2-chloroethyl)-2-methyl-6,7,8,9-tetrahydro-4H-pyrido[1,2-a]pyrimidin-4-one are placed, then a sodium carbonate solution or suspension (dissolved or suspended 8.5 g of sodium carbonate in 25 ml water) is added. The mixture is put into heating bath at 130-140° C. with stirring for 40 min, then cooled with continuous stirring to the room temperature and the precipitate solid is filtered, washed with pure water, and dried to give 3.75 g of the product in 91.5% yield. The product is purified to obtain a purity of 99.5% (determined by HPLC) with DMF and isopropanol.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A process for the preparation of risperidone-chemical named by 3-[2-[4-(6-fluoro-1,2-benzisoxazol-3-yl)piperidin-1-yl]ethyl]-2-methyl-6,7,8,9-tetrahydro-4H-pyrido[1,2-a]pyrimidin-4-one of formula (I): 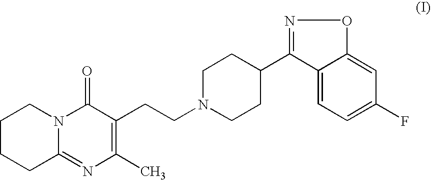
wherein the risperidone of formula (I) is prepared by condensing 6-fluoro-3-(4-piperidinyl)-1,2-benzisoxazole hydrochloride of formula (V) 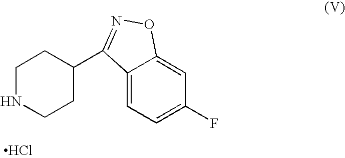
with 3-(2-chloroethyl)-2-methyl-6,7,8,9-tetrahydro-4H-pyrido[1,2-a]pyrimidin-4-one of formula (IV) as raw materials in a basic aqueous solution or suspension.

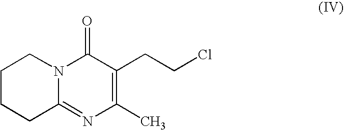

2. A process for preparing risperidone, according to claim 1,
wherein the raw material 6-fluoro-3-(4-piperidinyl)-1,2-benzisoxazole hydrochloride of formula (V): 3-(2-chloroethyl)-2-methyl-6,7,8,9-tetrahydro-4H-pyrido[1,2-a]pyrimidin-4-one of formula (IV) by molar ratio is within the range from 1:1.0 to 1:2.0;
wherein the basic aqueous solution or suspension is an aqueous solution or suspension of alkaline metal carbonates, and the content of alkaline metal carbonates in the basic aqueous solution or suspension is ranging from 15% to 40%; wherein the basic aqueous solution or suspension is employed in an amount ranging from 5 to 18 ml based on per gram of 6-fluoro-3-(4-piperidinyl)-1,2-benzisoxazole hydrochloride of formula (V);
wherein the reaction temperature is ranging from 101° C. to 140° C.;
wherein the reaction time is ranging from 10 min to 2 h.

3. A process for preparing risperidone, according to claim 1,
wherein the raw material 6-fluoro-3-(4-piperidinyl)-1,2-benzisoxazole hydrochloride of formula (V): 3-(2-chloroethyl)-2-methyl-6,7,8,9-tetrahydro-4H-pyrido[1,2-a]pyrimidin-4-one of formula (IV) by molar ratio is within the range from 1:1.1 to 1:1.3.

4. A process for preparing risperidone, according to claim 1, wherein the basic aqueous solution or suspension is an aqueous solution or suspension of alkaline metal carbonates, and the content of alkaline metal carbonates in the basic aqueous solution or suspension is 25%.

5. A process for preparing risperidone, according to claim 1, wherein the basic aqueous solution or suspension is employed in an amount ranging from 7 to 11 ml, based on per gram of 6-fluoro-3-(4-piperidinyl)-1,2-benzisoxazole hydrochloride of formula (V).

6. A process for preparing risperidone, according to claim 1, wherein alkaline metal carbonates in the basic aqueous solution or suspension is selected from sodium carbonate, potassium carbonate and a mixture thereof.

7. A process for preparing risperidone, according to claim 1, wherein alkaline metal carbonates in the basic aqueous solution or suspension is sodium carbonate.

8. A process for preparing risperidone, according to claim 1, wherein the reaction temperature is ranging from 101° C. to 130° C.

9. A process for preparing risperidone, according to claim 1, wherein the reaction time is ranging from 15 min to 1 h.
